(19) United States
(12) Patent Application Publication
(10) Pub. No.: US 2015/0100067 A1
(43) Pub. Date: Apr. 9, 2015

(54) METHODS AND SYSTEMS FOR COMPUTER-GUIDED PLACEMENT OF BONE IMPLANTS

(71) Applicant: University of Washington through its Center for Commercialization, Seattle, WA (US)

(72) Inventors: Peter R. Cavanagh, Seattle, WA (US); Ian Donaldson, Seattle, WA (US)

(21) Appl. No.: 14/509,873

(22) Filed: Oct. 8, 2014

Related U.S. Application Data
(60) Provisional application No. 61/888,151, filed on Oct. 8, 2013.

(51) Int. Cl. A61B 19/00; A61B 5/06

(52) U.S. Cl. CPC A61B 19/2203; A61B 19/5244; A61B 5/064; A61B 2019/5276; A61B 2019/5236; A61B 2019/524; Y10S 901/09; Y10S 901/46
USPC 606/130; 901/9; 901/46

(57) ABSTRACT

The present technology relates generally to systems and methods for computer-guided placement of bone implants. In some embodiments, for example, a method of computer-guided surgical insertion of an implant into a target bone includes imaging the target bone to obtain three-dimensional (3D) image data, and, based on the 3D image data, determining an entry point and trajectory for insertion of the implant. The method also includes mapping the 3D image data, the entry point, and the trajectory into a surgical field, followed by instructing a clinician to insert the implant into the target bone based on the determined entry point and trajectory.

### CROSS-REFERENCE TO RELATED APPLICATION

This application claims the benefit of U.S. Provisional Patent Application No. 61/888,151, filed Oct. 8, 2013, which is incorporated herein by reference in its entirety.

### TECHNICAL FIELD

The present technology is generally related to methods and systems for optimizing computer-guided surgery involving insertion of bone implants. In particular, several embodiments are directed to methods and systems for optimizing entry point and trajectory for bone implants.

### BACKGROUND

Insertion of implants into bone—for example percutaneous insertion of fixation screws—is a common surgical procedure. Typically, a surgeon manually drills a K-wire into the bone with visual guidance from intraoperative 2-dimensional fluoroscopic imaging. The implant (e.g., a bone screw) is then advanced over the K-wire and drilled into the bone for stabilization. Major complications can result from inaccurate K-wire placements. Achieving accurate placement of the K-wire can be particularly difficult in smaller or irregularly shaped bones requiring fixation, for example the scaphoid or other bones in the hand, foot, or spine. Two major complications observed with scaphoid fixation include violation of the cortex surface and post-operative scaphoid non-union (in which the bone fails to re-fuse into a single body). These complications can be attributed at least in part to non-optimal screw placement, as prominent hardware irritates articular cartilage and centralized longitudinal screw placement is advantageous in encouraging bone union. Additionally, manual placement of K-wires often requires multiple attempts before the hardware is successfully inserted. Multiple attempts detract from operating room efficiency and can also threaten the structural integrity of the target bone, potentially leading to further complications. Accordingly, there is a need for improved systems and methods for optimizing insertion of bone implants.

### BRIEF DESCRIPTION OF THE DRAWINGS

FIG. 1 is a schematic illustration of a system for computer-guided placement of a bone implant according to one embodiment of the present technology.

FIG. 2 is a flow diagram of a method for computer-guided placement of a bone implant according to one embodiment of the present technology.

FIG. 3A illustrates a system for obtaining a three-dimensional image of a target bone according to one embodiment of the present technology.

FIG. 3B illustrates three-dimensional image data of the target bone of FIG. 3A.

FIG. 4A illustrates a flow diagram of a method for optimizing entry point and trajectory for insertion of a bone implant according to one embodiment of the present technology.

FIG. 4B illustrates the optimized entry point and trajectory for insertion of a bone implant according to one embodiment of the present technology.

FIG. 5A illustrates a system for registering the surgical field according to one embodiment of the present technology.

FIG. 5B illustrates a graphical representation of mapping the 3D image data into the surgical field.

FIG. 6A illustrates a system for positioning surgical equipment at an optimized entry point and trajectory according to one embodiment of the present technology.

FIG. 6B illustrates another system for positioning surgical equipment at an optimized entry point and trajectory according to one embodiment of the present technology.

### DETAILED DESCRIPTION

As noted above, there are myriad problems with manual placement of K-wires to guide implants, particularly in small or irregularly shaped bones such as those of the hands, feet, and spine. These problems can be addressed with the use of computer-assisted surgery including a navigation system to guide manual or robotic placement of K-wires or other guiding elements. One example of such a navigation system is a vision system having high sensitivity cameras configured to track reflective or actively illuminated objects (called targets) through space or to form images of surfaces as probes with targets attached are traced over them.

In some embodiments, a system for computer-assisted insertion of bone implants couples a surgical navigation system, 3D imaging, and a robotic arm to dynamically guide K-wire placement during insertion of bone implants, for example scaphoid fixation with a compression screw. This approach provides several benefits over prior techniques. First, surgeons will no longer need to interpret fluoroscopy images intraoperatively or perform manual K-wire alignment; instead, trajectories can be computed automatically and K-wire alignment can be performed robotically or under computer-assisted guidance. Secondly, target bone shape data obtained by CT or other imaging can be used to calculate an optimized screw trajectory, surface entry point, screw length and screw diameter; these parameters may then be applied in the procedure. Taken together, these innovations mitigate the difficulty of manual K-wire placement and yield optimized implant placements in target bones.

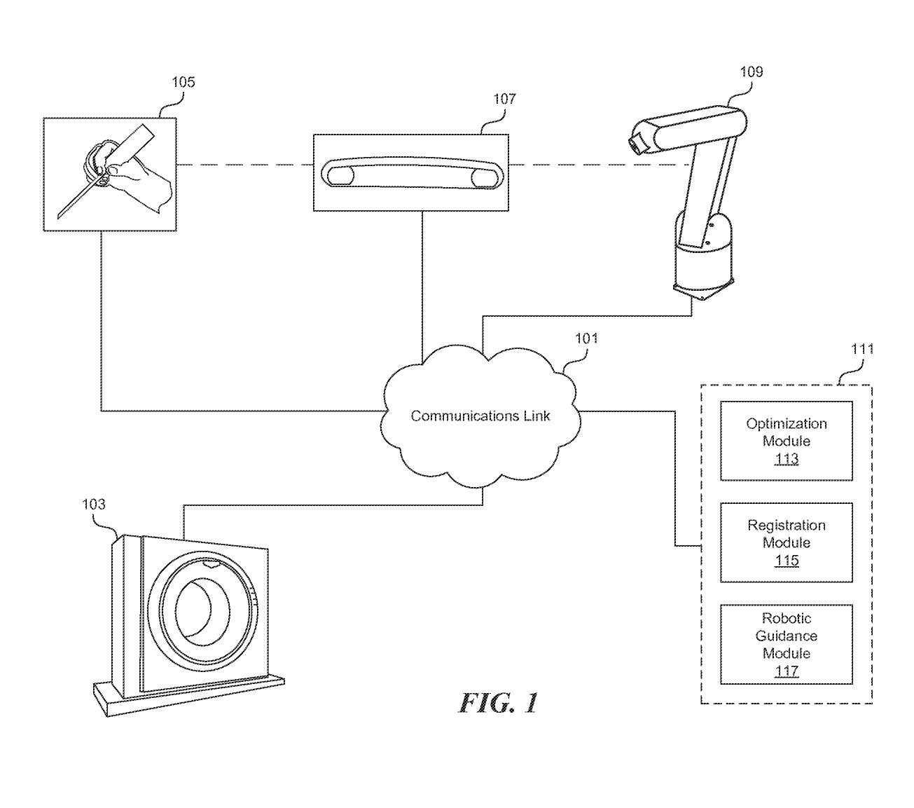

Specific details of several embodiments of the present technology are described below with reference to FIGS. 1-6. Although many of the embodiments are described below with respect to devices, systems, and methods for computer-guided insertion of bone implants, other embodiments are within the scope of the present technology. Additionally, other embodiments of the present technology can have different configurations, components, and/or procedures than those described herein. For example, other embodiments can include additional elements and features beyond those described herein, or other embodiments may not include several of the elements and features shown and described herein.

For ease of reference, throughout this disclosure identical reference numbers are used to identify similar or analogous components or features, but the use of the same reference number does not imply that the parts should be construed to be identical. Indeed, in many examples described herein, the identically numbered parts are distinct in structure and/or function.

### Selected Embodiments of Systems and Methods for Computer-Guided Placement of Bone Implants

FIG. 1 is a schematic illustration of a system for computer-guided placement of a bone implant according to one embodiment of the present technology. The system includes a number of components in communication with one another via communication link 101 which can be, for example, a public internet, private network such as an intranet, or other network. Connection between each component and the communication link 101 can be wireless (e.g., WiFi, Bluetooth, NFC, GSM, cellular communication such as CDMA, 3G, or 4G, etc.) or wired (e.g., Ethernet, FireWire cable, USB cable, etc.). An imaging component 103 is coupled to the communication link 101. In some embodiments, the imaging component 103 can be configured to obtain three-dimensional (3D) images of a target bone. For example, in some embodiments the imaging component 103 can be a magnetic resonance imaging (MRI) machine, a computed tomography (CT) machine, a 3D ultrasound machine, or other device configured to obtain 3D images of a target bone. A navigation probe 105 is in communication with communications link 101 as well as a camera 107 configured to detect the location of the navigation probe 105. For example, the camera 107 can optically track the movement of the navigation probe 105 through 3D space with high resolution. In some embodiments, the camera 107 can be configured to track the movement of navigation probe 105 using other techniques, for example sonic or electromagnetic detection. By tracing the navigation probe 105 over a surface of the target bone, the position and orientation of the target bone in a surgical field can be determined This can facilitate registration of the 3D image obtained via imaging component 103 into a surgical field. This arrangement can also allow the positioning of a K-wire, drill guide, or other surgical equipment adjacent the target bone at the desired position and orientation once the optimization has been completed. A robotic arm 109 is also coupled via communications link 101 to the other component. The robotic arm 109 can also be configured to position a K-wire, drill guide, or other surgical equipment adjacent the target bone at the desired position and orientation. In some embodiments, the camera 107 can be configured to track the position of the robotic arm 109 in the surgical field.

A computing component 111 includes a plurality of modules for interacting with the other components via communications link 101. The computing component 111 includes, for example, an optimization module 113, a registration module 115, and a robotic guidance module 117. In some embodiments, the computing component 111 can include a processor such as a CPU which can perform operations in accordance with computer-executable instructions stored on a computer-readable medium. In some embodiments, the optimization module, registration module, and robotic guidance module may each be implemented in separate computing devices each having a processor configured to perform operations. In some embodiments, two or more of these modules can be contained in a single computing device.

The optimization module 113 can be configured to receive 3D image data of the target bone via communications link 101. The 3D image data may be obtained via imaging component 103. Generation of suitable 3D image data generally requires the segmentation of the raw image using image analysis software. The optimization module 113 can analyze the 3D image data to determine an optimal entry point and trajectory for an implant to be inserted into the target bone, as described in more detail below. In some embodiments, the 3D image data and the determined optimal entry point and trajectory data can be in a reference frame. Registration module 115 can be mapped into a surgical reference frame. The surgical reference frame can be determined using the navigation probe 105 and the camera 107, as discussed below. Robotic guidance module 117 can communicate with the robotic arm 109 and cause the robotic arm 109 to move to a desired position in the surgical field.

Those of ordinary skill in the art will appreciate that the routines and other functions and methods described herein can be performed by various processing devices, such as the computing component 111 or one or more of the modules 113, 115, 117. The processes can be implemented as an application specific integrated circuit (ASIC), by a digital signal processing (DSP) integrated circuit, through conventional programmed logic arrays or circuit elements. While many of the embodiments can be implemented in hardware (e.g., one or more integrated circuits designed specifically for a task), such embodiments could equally be implemented in software and be performed by one or more processors. Such software can be stored on any suitable computer-readable medium, such as microcode stored in a semiconductor chip, on a computer-readable disk, or downloaded from a server and stored locally at a client.

The computing component 111, optimization module 113, registration module 115, and/or robotic guidance module 117 may each include one or more central processing units or other logic-processing circuitry, memory, input devices (e.g., keyboards and pointing devices), output devices (e.g., display devices and printers), and storage devices (e.g., magnetic, solid state, fixed and floppy disk drives, optical disk drives, etc.). Such devices may include other program modules such as an operating system, one or more application programs (e.g., word processing or spread sheet applications), and the like. The computers may be general-purpose devices that can be programmed to run various types of applications, or they may be single-purpose devices optimized or limited to a particular function or class of functions. Aspects of the p may be practiced in a variety of other computing environments.

The communications link 101 can be the Internet, or a private network, such as an intranet may likewise. The network may have a client-server architecture, in which a computer is dedicated to serving other client computers, or it may have other architectures such as peer-to-peer, in which one or more computers serve simultaneously as servers and clients. Also, various communication channels, such as local area networks, wide area networks, or point-to-point dial-up connections, may be used instead of the Internet. The system may be conducted within a single computer environment, rather than a client/server environment. Also, the computing component and/or modules may comprise any combination of hardware or software.

Although not required, aspects of the present technology are described in the general context of computer-executable instructions, such as routines executed by a general-purpose data processing device. Those skilled in the relevant art will appreciate that aspects of the present technology can be practiced with other communications, data processing, or computer system configurations, including Internet appliances, hand-held devices, wearable computers, multi-processor systems, microprocessor-based or programmable consumer electronics, network PCs, mini-computers, mainframe computers, and the like. Any type computer-readable media that can store data accessible by a processor may be used, such as magnetic hard and floppy disk drives, optical disk drives, magnetic cassettes, tape drives, flash memory cards, digital video disks (DVDs), Bernoulli cartridges, RAMs, ROMs, smart cards, etc. Indeed, any medium for storing or transmitting computer-readable instructions and data may be employed, including a connection port to a network such as a local area network (LAN), wide area network (WAN) or the Internet. The terms “memory” and “computer-readable storage medium” include any combination of temporary, persistent, and/or permanent storage, e.g., ROM, writable memory such as RAM, writable non-volatile memory such as flash memory, hard drives, solid state drives, removable media, and so forth, but do not include a propagating signal per se.

Aspects of the present technology can be embodied in a special purpose computer or data processor that is specifically programmed, configured, or constructed to perform one or more of the computer-executable instructions explained in detail herein. While aspects of the present technology, such as certain functions, are described as being performed exclusively on a single device, the present technology can also be practiced in distributed environments where functions or modules are shared among disparate processing devices, which are linked through a communications network, such as a Local Area Network (LAN), Wide Area Network (WAN), or the Internet. In a distributed computing environment, program modules may be located in both local and remote memory storage devices.

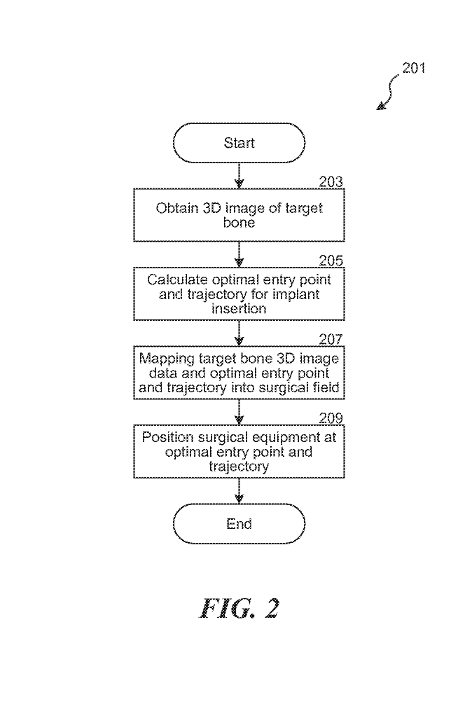

FIG. 2 is a flow diagram of a method for computer-guided placement of a bone implant according to one embodiment of the present technology. The routine 201 begins in block 203 with obtaining a 3D image of a target bone. This may include a numerical representation of the bone obtained from image analysis software. The target bone is the bone in which an implant is to be inserted. For example, in some embodiments the target bone can be a small bone of the hand, foot, or spine. In some embodiments, the target bone can be a scaphoid—one of the carpal bones of the wrist or a small bone of the foot such as a metatarsal bone. The implant to be inserted can be a bone screw, bone plate, or other implant configured to be inserted into a target bone. The 3D image of the target bone can be obtained via a variety of approaches, for example MRI, CT, or 3D ultrasound. Imaging of the target bone and subsequent use of image analysis software can provide 3D image data for analysis and computation.

The routine 201 continues in block 205 with calculating the optimal entry point and trajectory for implant insertion. For example, the 3D image data obtained in block 201 can be analyzed to calculate an optimal entry point and trajectory. In some embodiments, an optimization algorithm can be utilized to calculate an optimal entry point and trajectory. In some embodiments, the optimization algorithm includes a cost function and one or more constraints. For example, the cost function (or objective function) can minimize the cumulative distance between each point on a selected area on the surface of the bone and a longitudinal vector of the implant. Other cost functions can be defined to maximize purchase of the bone implant, or to otherwise achieve a desired position of the implant within the target bone. In some embodiments, the optimization algorithm can constrain the position so that the implant does not penetrating the outer cortex of the target bone. In some embodiments, the optimization algorithm can constrain the position so that the implant crosses a fracture line in the target bone. In some embodiments, the insertion point for the implant can be constrained to a particular location on the target bone (for example the dorsal surface of the scaphoid or the accessible regions of the base of the fifth metatarsal). Any number of constraints can be included in the optimization algorithm depending on the target bone, the type of implant, etc. In some embodiments, a user can provide a starting point for the optimization algorithm, for example an approximate estimation of the optimal entry point and trajectory for the implant. In other embodiments, no starting point is provided. The optimization algorithm utilizes the 3D image data and determines an optimal entry point and trajectory for implant insertion. As used herein, “optimal” includes both the global optimum and local optima, with the former generally being the desirable condition.

The 3D data and the determined optimal entry point and trajectory can be provided in a reference frame. In block 207 the target bone 3D image data and optimal entry point and trajectory are mapped from the reference frame into a surgical field. The surgical field is a frame of reference which includes the position of target bone during surgery. To obtain surgical field data, at least a portion of the target bone can be detected while in position for surgery. For example, once the patient is in the operating table and is stabilized for surgery, the position of the target bone can be obtained via a number of approaches. In some embodiments, a navigation probe can be traced over an exposed surface of the target bone. An associated camera can track the position of the navigation probe as it is traced over the surface of the target bone, and thereby determine the position of the traced surface in the surgical frame. In some embodiments, reference points such as tantalum markers can be inserted or applied to the target bone. Imaging of the target bone in the surgical frame can utilize these reference points to map the 3D image data (which can also detect the reference points) into the surgical field. In some embodiments, rather than artificial reference points such as tantalum markers, anatomical features of the bone can be utilized, for example bony landmarks detectable via imaging or otherwise. Once a portion of the target bone has been detected in the surgical field, that detected portion can be compared to the 3D image data. Based on this comparison, the 3D image data can be mapped from its reference frame into the surgical field. In some embodiments, a user can manually aid in the mapping process. For example, a graphical depiction of the detected portion of the target bone can be displayed in a surgical field. A graphical depiction of the 3D image data and the determined optimal entry point and trajectory can be displayed as overlapping with the graphical depiction of the target bone in the surgical field. The user can then rotate and otherwise manipulate the position and orientation of the 3D image data until it corresponds to the portion of the target bone detected in the surgical field. Once the two graphical representations correspond, the 3D image data (and the determined optimal entry point and trajectory) have been registered or mapped to the surgical field. In some embodiments, a user can provide an initial approximate alignment, followed by a computational alignment to achieve more precise mapping.

The routine 201 continues in block 209 with positioning surgical equipment at the optimal entry point and trajectory. In some embodiments, the surgical equipment can be a drill guide with attached targets or a K-wire, which can define the orientation and entry point for drilling or advancement of other tools to the target bone. In some embodiments, a surgical robot can automatically position a K-wire, drill guide, or other surgical equipment at the determined optimal entry point and trajectory. For example, the surgical robot can be instructed to move in the surgical field, and so can utilize the 3D image data and optimal entry point and trajectory (which have been mapped into the surgical field) to move the surgical equipment to the appropriate position and orientation. In some embodiments, the surgical robot can include one or more targets and can be tracked by a camera to detect its position in the surgical field. In some embodiments, a surgeon or other clinician can manually position the surgical equipment at the optimal entry point and trajectory. In some embodiments, the surgical equipment can be associated with one or more targets whose position can be tracked in the surgical field. As the surgeon positions the surgical equipment nearer to the target bone, a feedback system can indicate whether the surgical equipment is at or near the optimal entry point and trajectory. For example, a graphical display can indicate where the surgical equipment is positioned with respect to the determined optimal entry point and trajectory. In some embodiments, feedback can include auditory, visual, haptic, or other feedback to the surgeon to indicate the position of the surgical equipment with respect to the optimal entry point and trajectory. Once at or sufficiently near the optimal entry point and trajectory, the feedback system can indicate correct placement to the surgeon.

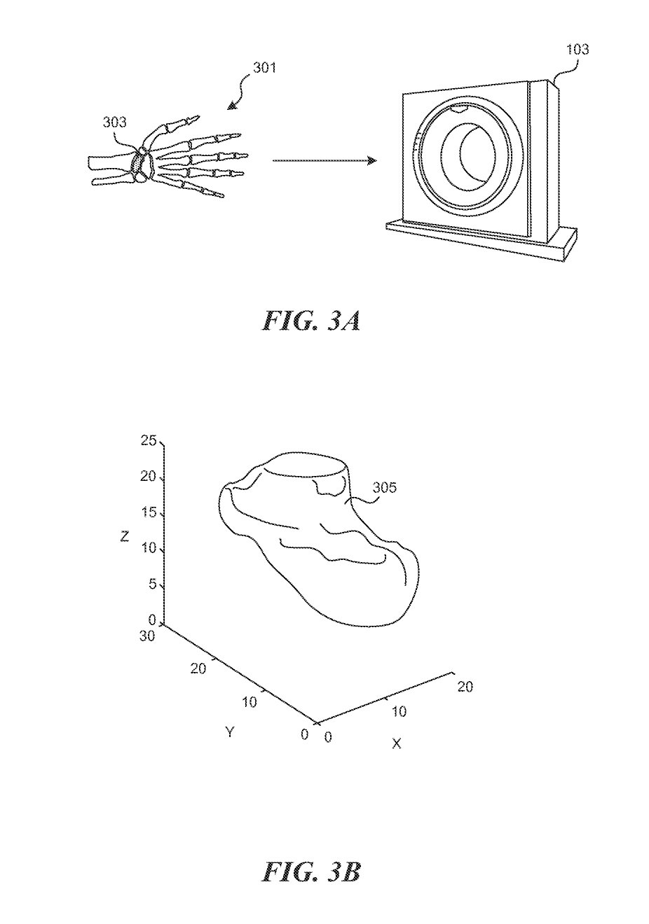

FIG. 3A illustrates a system for obtaining a three-dimensional image of a target bone according to one embodiment of the present technology. In the illustrated embodiment, the target bone is a scaphoid. The hand 301 can be positioned with respect to an imaging component 103 so as to obtain a 3D image of the target bone 303. FIG. 3B illustrates three-dimensional image data of the target bone of FIG. 3A. As noted above, in other embodiments the target bone can take other forms, for example other bones in the hand, or bones of the foot or spine. The 3D image of the target bone can be obtained via a variety of approaches, for example MRI, CT, or 3D ultrasound. In some embodiments, the 3D image data of the target bone can be presented as a graphical representation 305 as in FIG. 3B. In some embodiments a user can manipulate the graphical representation, for example rotating, scaling, etc. Image analysis software can be used to segment the images and provide 3D coordinates of the bone of interest.

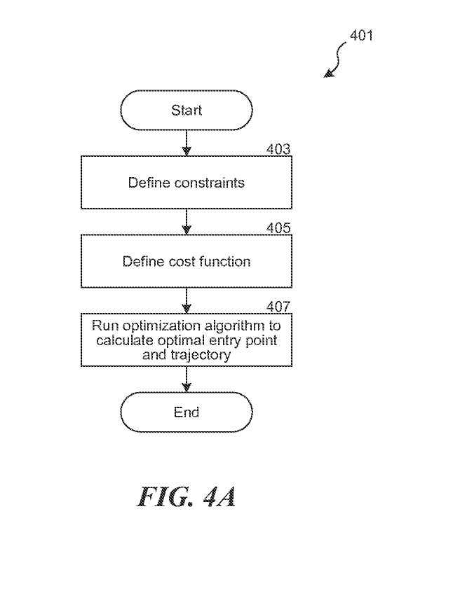

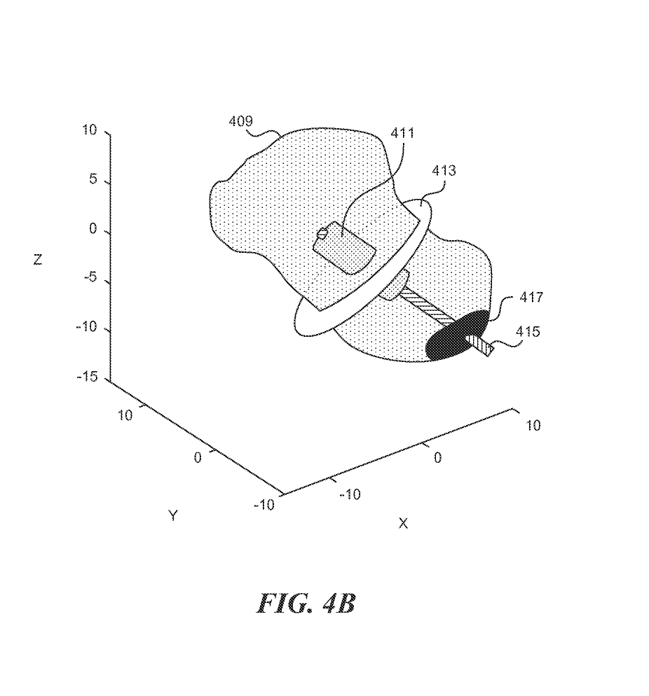

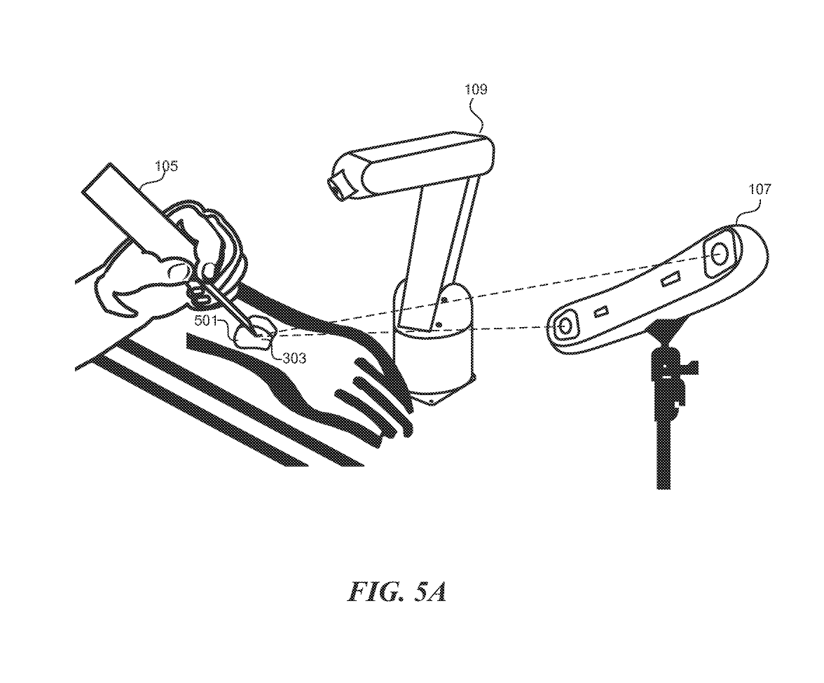

FIG. 4A illustrates a flow diagram of a method for optimizing entry point and trajectory for insertion of a bone implant according to one embodiment of the present technology. The routine 401 begins in block 403 with defining constraints for an optimization algorithm. In some embodiments, the optimization algorithm can constrain the position of the implant so that the implant does not penetrating the outer cortex of the target bone. In some embodiments, the optimization algorithm can constrain the position of the implant so that the implant crosses a fracture line in the target bone. In some embodiments, the insertion point for the implant can be constrained to a particular location on the target bone (for example the dorsal surface of the scaphoid). In some embodiments, the implant can be constrained so that at least a pre-defined portion (e.g. 25% of the length) lies on either side of the fracture. Any number of constraints can be included in the optimization algorithm depending on the target bone, the type of implant, etc.

The routine 401 continues in block 405 with defining the cost function. A variety of cost functions are possible, for example the cost function can minimize the cumulative distance between each point on a selected surface of the bone and a longitudinal vector of the implant. In some embodiments, the cost function can maximize purchase of the bone implant, or to otherwise achieve a desired position of the implant within the target bone. In some embodiments, the cost function can minimize the cumulative distance between the screw and a pre-defined target zone (e.g., a scaled-down version of the target bone, which can provide for an additional safety buffer for positioning the implant). In some embodiments, the cost function can incorporate two or more of these objectives to determine an optimum entry point and trajectory. The routine 401 continues in block 407 with running the optimization algorithm to calculate the optimal entry point and trajectory. The optimization algorithm can utilize 3D image data of the target bone to determine an optimal entry point and trajectory for implant insertion. In some embodiments, a user can provide a starting point for the optimization algorithm, for example an approximate estimation of the optimal entry point and trajectory for the implant. In other embodiments, no starting point is provided.

FIG. 4B illustrates the optimized entry point and trajectory for insertion of a bone implant according to one embodiment of the present technology. This graphical representation of the optimal entry point and trajectory illustrates the output of the optimization algorithm of FIG. 4A. The target bone 409 is in this embodiment a scaphoid. As noted above, in other embodiments the target bone can be another bone in the body. A fracture plane 413 indicates the position of a fracture. In some embodiments, the fracture plane 413 can be automatically identified by analyzing the 3D image data of the target bone. In some embodiments, a user can manually position the fracture plane 413 with respect to the graphical representation of the target bone 409. As noted above, in some embodiments the optimization algorithm can rely on the position of the fracture to determine the optimal entry point and trajectory. The implant 411 is illustrated in this embodiment as a screw positioned across the fracture plane 413. In other embodiments the implant can take other forms, for example a bone plate, rod, or other implant configured to be inserted into a target bone. The determined trajectory 415 indicates the axis along which the implant 411 is to be implanted. An entry region 417 is shown, with the trajectory 415 intersecting the entry region 417. In some embodiments, the entry region 417 can be one of the constraints of the optimization algorithm, i.e., the optimization algorithm is constrained so that the determined trajectory passes through the entry region 417. In some embodiments the entry region can be defined manually by a user.

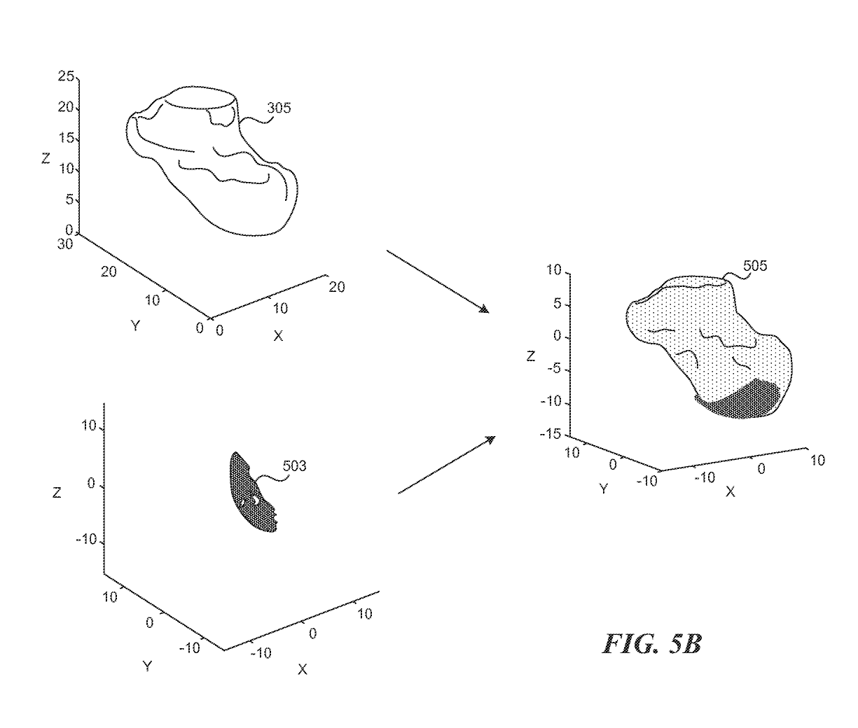

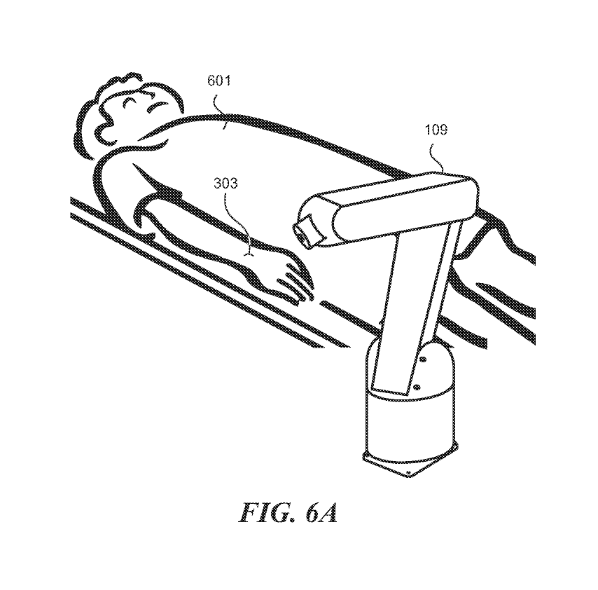

FIG. 5A illustrates a system for registering the surgical field, and FIG. 5B illustrates a graphical representation of mapping the 3D image data into the surgical field. Referring to FIGS. 5A and 5B together, a navigation probe 105 is traced over the exposed surface 501 of the target bone 303, which in this case is a scaphoid. A surgical robot 109 can also be positioned adjacent the target bone 303, and can be tracked by the camera 107. As the navigation probe 105 is traced over the surface 501, camera 107 detects the position of the navigation probe 105 in the surgical field. This detected position can be used to generate a graphical representation 503 of the portion of the target bone 303. In some embodiments, other reference points on the target bone can be detected, for example artificial reference points such as tantalum markers, or anatomical reference markers such as bony landmarks. Based on data from the camera 107, the location of the surface 501 in the surgical field is obtained. In some embodiments, this location can be obtained by a process of optimization. Once a portion of the target bone 303 has been detected in the surgical field, that detected portion can be compared to the 3D image data of the entire target bone. For example, the graphical representation 305 of the 3D image data of the target bone can be compared with the graphical representation 505 of the portion of the target bone in the surgical field. By comparing these two, the target bone can be mapped onto the surgical field such that the detected portion of the target bone (graphical representation 505) overlaps with its corresponding portion of the 3D image data (graphical representation 305). A graphical representation 505 of the overlapping portions is illustrated.

In some embodiments, a transformation matrix can be used to register the 3D image data (including the determined optimal entry point and trajectory) into the surgical field. In some embodiments, a user can manually aid in the mapping process. For example, a graphical depiction of the detected portion of the target bone can be displayed in a surgical field. A graphical depiction of the 3D image data and the determined optimal entry point and trajectory can be displayed as overlapping with the graphical depiction of the target bone in the surgical field. The user can then rotate and otherwise manipulate the position and orientation of the 3D image data until it corresponds to the portion of the target bone detected in the surgical field. Once the two graphical representations correspond, the 3D image data (and the determined optimal entry point and trajectory) have been registered to the surgical field. In some embodiments, a user can provide an initial approximate alignment, followed by a computational alignment to achieve more precise mapping.

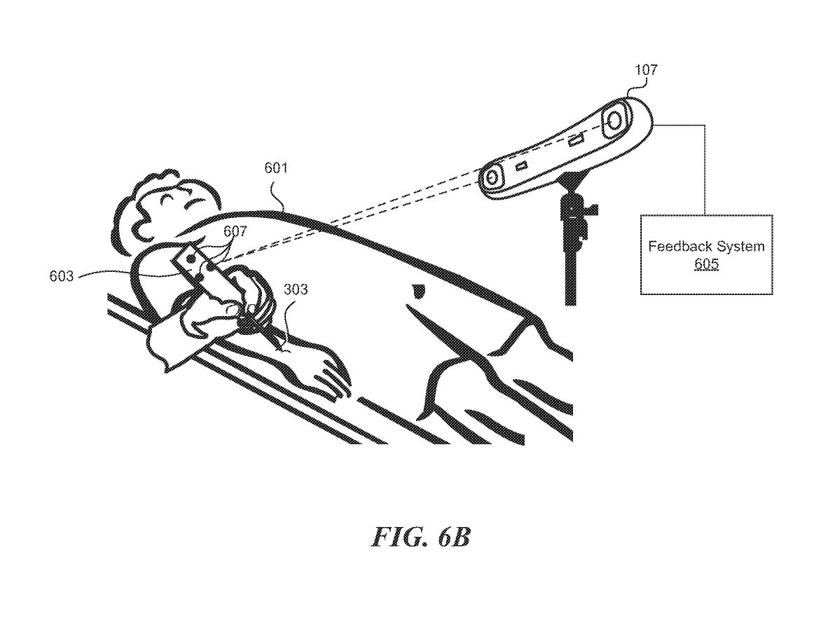

FIG. 6A illustrates a system for positioning surgical equipment at an optimized entry point and trajectory according to one embodiment of the present technology. As illustrated, a surgical robot 109 is positioned with respect to a patient 601 so as to position surgical equipment adjacent to the target bone 303, which in this instance is a scaphoid. In some embodiments, the surgical robot 109 can be fitted with a drill guide or K-wire, and can position the drill guide or K-wire adjacent the target bone 303 so as to define the orientation and entry point for drilling or advancement of other tools to the target bone 303. For example, the surgical robot can be instructed to move in the surgical field, and so can utilize the 3D image data and optimal entry point and trajectory (which have been mapped into the surgical field) to move the surgical equipment to the appropriate position and orientation. In some embodiments, the surgical robot can include one or more targets and can be tracked by a camera to detect its position in the surgical field. In some embodiments, the surgical robot can also insert the implant into the target bone. For example, the surgical robot can position a drill guide against the bone, followed by drilling along the determined trajectory and entry point to a desired depth. The surgical robot may then insert the screw or other implant into the target bone.

Referring to FIG. 6B, in some embodiments, rather than using a surgical robot, a surgeon or other clinician may manually can manually position the surgical equipment at the optimal entry point and trajectory. For example, a surgical equipment 603 (such as a K-wire, drill guide, etc.) can be associated with one or more targets 607 whose position can be tracked in the surgical field by a camera 107. A feedback system 605 can indicate the position of the surgical equipment 603 with respect to the determined entry point and trajectory of the target bone 303. In some embodiments, feedback can include auditory, visual, haptic, or other feedback to the surgeon to indicate the position of the surgical equipment with respect to the optimal entry point and trajectory. Once the surgical equipment is positioned at or sufficiently near the optimal entry point and trajectory, the feedback system 605 can indicate correct placement to the surgeon.

### CONCLUSION

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

From the foregoing, it will be appreciated that specific embodiments of the present technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. A method of computer-guided surgical insertion of an implant into a scaphoid positioned in a surgical field, the method comprising:
receiving three-dimensional (3D) image data of the scaphoid;
based on the 3D image data, automatically determining an optimal entry point and trajectory for insertion of the implant into the scaphoid;
mapping the 3D image data, the entry point, and the trajectory into the surgical field; and
robotically positioning a guiding element adjacent to the scaphoid based on the determined entry point and trajectory.

2. The method of claim 1 wherein the 3D image data of the scaphoid comprises 3D image data obtained via at least one of: computed tomography (CT), magnetic resonance imaging (MRI), and ultrasound.

3. The method of claim 1 wherein determining the entry point and the trajectory comprises non-linear constrained optimization of the position and entry point of the implant with respect to the scaphoid.

4. The method of claim 1 wherein mapping the 3D image data, the entry point, and the trajectory into the surgical field comprises:
detecting a position of a portion of the scaphoid in the surgical field; and
based on the detected position of the portion of the scaphoid, mapping the 3D image data, the entry point, and the trajectory into the surgical field.

5. The method of claim 4 wherein detecting the position of the portion of the target bone comprises tracing a surface of the target bone with an optical probe.

6. The method of claim 4 wherein detecting the position of the portion of the target bone comprises detecting a position of one or more fiducial markers in or on the target bone

7. A method of computer-guided surgical insertion of an implant into a target bone positioned in a surgical field, the method comprising:
receiving three-dimensional (3D) image data of the target bone;
based on the 3D image data, automatically determining an entry point and trajectory for insertion of the implant into the target bone;
mapping the 3D image data, the entry point, and the trajectory into a surgical field; and
instructing a clinician to insert the implant based on the determined entry point and trajectory.

8. The method of claim 7 wherein the target bone comprises a bone of the hand, foot, or spine, and wherein the implant comprises a bone screw.

9. The method of claim 7 wherein determining the entry point and the trajectory comprises non-linear constrained optimization of the position and entry point of the implant with respect to the target bone.

10. The method of claim 9 wherein the non-linear constrained optimization of the position of the implant is configured to maximize purchase of the implant within the target bone.

11. The method of claim 7 wherein mapping the 3D image data, the entry point, and the trajectory in the surgical field comprises:
detecting a position of a portion of the target bone in the surgical field; and
based on the detected position of the portion of the target bone, mapping the 3D image data, the entry point, and the trajectory in the surgical field.

12. The method of claim 11 wherein detecting the position of the portion of the target bone comprises tracing a surface of the target bone with an optical probe.

13. The method of claim 11 wherein detecting the position of the portion of the target bone comprises detecting a position of one or more fiducial markers in or on the target bone

14. The method of claim 13 wherein the one or more fiducial markers comprise anatomical structures of the target bone.

15. The method of claim 13 wherein the one or more fiducial markers comprise artificial markers disposed in or on the target bone.

16. The method of claim 7 wherein instructing a clinician to insert the implant based on the determined entry point and trajectory comprises robotically positioning a guiding element adjacent the target bone at the desired entry point and trajectory.

17. The method of claim 7 wherein instructing a clinician to insert the implant based on the determined entry point and trajectory comprises: providing feedback to the clinician regarding the position of a guiding element with respect to the target bone and the determined entry point and trajectory.

18. A system for computer-guided surgical insertion of an implant into a target bone in a surgical field, the system comprising:
an imaging component configured to obtain three-dimensional (3D) image data of the target bone;
an optimization module comprising a computing device having a processor, the processor configured to perform operations comprising
based on the 3D image data of the target bone, determining an optimal entry point and trajectory for insertion of the implant into the target bone; and
a registration module comprising a computing device having a processor, the processor configured to perform operations comprising
mapping the 3D image data, the entry point, and the trajectory into the surgical field.

19. The system of claim 18, further comprising:
a movable surgical robot coupled to a guiding element; and
a surgical guidance module comprising a computing device having a processor, the processor configured to perform operations comprising
causing the surgical robot to position the guiding element adjacent to the target bone based on the determined entry point and trajectory.

20. The system of claim 18, further comprising:
a guiding element coupled to a position tracking system configured to track the position of the guiding element with respect to the determined entry point and trajectory; and
a surgical guidance module comprising a computing device having a processor, the processor configured to perform operations comprising
receiving the tracked position of the guiding element with respect to the determined entry point and trajectory; and
providing indicia of the position of the guiding element with respect to the determined entry point and trajectory.
